(19) United States
(12) Patent Application Publication
(10) Pub. No.: US 2006/0088602 A1
(43) Pub. Date: Apr. 27, 2006

(54) Electromagnetic field regulating compositions

(76) Inventors: George Cioca, Lake Grove, NY (US); Paolo Glacomoni, Commack, NY (US); Glen Rein, Huntington, NY (US); Vasile Ionita-Manzatu, Old Bethpage, NY (US); Abdul Manirazman, Port Jefferson, NY (US); Liliana George, Centerport, NY (US); Andrew Bevacqua, East Setauket, NY (US); Harvey Gedeon, New York, NY (US)

Correspondence Address: THE ESTEE LAUDER COS, INC, 125 PINELAWN ROAD, MELVILLE, NY, 11747, US

(21) Appl. No.: 10/970,313

(22) Filed: Oct. 21, 2004

(51) Int. Cl. A61K 33/26; A61K 33/24; A61K 33/34

(52) U.S. Cl. 424/634; 424/649; 424/646; 977/926

(57) ABSTRACT

A cosmetic or pharmaceutical emulsion composition that is topically applied to the skin to protect it against damage caused by electromagnetic field generated from everyday appliances such as televisions, computers and cellular phones. The compositions contain ambient electromagnetic wave dampening effective amounts of at least one single domain magnet as the sole magnetic component and a non-magnetic ionic component. The sole magnetic component works particularly well in the composition of the present invention when it comprises granatite and when the composition is an oil in water emulsion.

### FIELD OF THE INVENTION

The invention relates to topically applied electromagnetic field shielding cosmetic or pharmaceutical compositions. More specifically, the invention relates to such compositions containing a magnetic component and an ionic component capable of dampening the affect of an electromagnetic field on the skin.

### BACKGROUND OF THE INVENTION

In today's society televisions, computers, microwaves, and cellular phones are commonplace tools of everyday living. Appliances such as these and the power lines that supply them, however useful in communications and as technological vehicles for enhanced business and trade relations from the smallest town to the largest international commerce network, are not without their drawbacks. These devices emit electromagnetic waves which have been experimentally proven to be harmful to the physiological systems of the human body including skin cells. Oftedal, G., et al., “Long-term Effects on Symptoms by Reducing Electric Fields From visual Display Units”, Scand J Work Environ Health, vol. 25, no. 5, pp. 415-21 (1999); Johansson, O., et al., “Cutaneous Mast Cells Are Altered in Normal Healthy Volunteers Sitting in Front of Ordinary TVs/PCs—Results From Open-Field Provocation Experiments”, J Cutan Pathol, vol. 28, pp. 513-19 (2001). For example, it has recently been found that normal cutaneous mast cells can be altered after being exposed to electromagnetic fields (EMF) generated by video display terminals. In general, skin cells can be exposed to EMF generated by, for example, television, power lines and stations, and radio and microwave ovens, as they all emit very low frequencey (VLF), extremely low frequency (ELF, and radio frequency (RF) electromagnetic waves. It has also been noted that microwaves in the range of frequencies of cellular phones have been related to increased chromosomal aberrations in cultured human lymphocytes, as well as an increased release of histamine from mast cells.

Exposure to VLF and ELF electromagnetic waves is being recognized as a possible cause of mild skin disorders. The reaction on the skin to EMF exposure can be seen in, for example, rosacea-like symptoms including itching, warm sensations, pain, erythema, papules, and pustules. Oftedal, G., et al., “Long-term Effects on Symptoms by Reducing Electric Fields From visual Display Units”, Scand J Work Environ Health, vol. 25, no. 5, pp. 415-21 (1999); “Cutaneous Mast Cells Are Altered in Normal Healthy Volunteers Sitting in Front of Ordinary TVs/PCs—Results From Open-Field Provocation Experiments”, J Cutan Pathol, vol. 28, pp. 513-19 (2001). Further, 50 to 60 Hz EMF, the frequencies most likely to pose a threat to skin healthiness, have been found to exhibit specific biological effects. In particular, it has been found that this range of EMF affects intracellular calcium transport and induces calcium oscillations in cultured cells. Therefore, it is possible that EMF could interfere with calcium-induced differentiation of keratinocytes, and therefore, result in the provocation of cellular necrosis causing inflammation and/or irritation.

In addition to the general population that may be prone to react to EMF exposure, in particular, there is a phenomenon being introduced as “screen dermatitis” where individuals possess certain histological features such as high levels of histamine-positive mast cells and somatostatin-positive dendritic cells. Although total shielding against electromagnetic radiation is not necessary, since many sources of radiation are not believed to be inherently harmful, skin symptons associated with exposure to electric fields can be reduced with two basic types of filtering systems. The first type of filter blocks the emission of radiation at the source. The other type of filter is applied to the target in need of protection. As more is learned about the effects of radiation, products that can be topically applied on the surface of the skin for the purpose of shielding and/or dampening the intensity of VLF, ELF or RF are of interest to the anyone seeking this type of protection. Thus, in a technologically developed society that depends heavily on devices that emit VLF or ELF or RF the need for protection is expected to increase. Presently, protection is available in the form of protective clothing to shield against electromagnetic radiation, as for example, a textile fabric disclosed in U.S. Pat. No. 5,103,504. It is also known to intercept the electromagnetic radiation at the source, by shielding electromagnetic radiation emitted by the device itself. For example, U.S. Pat. No. 6,001,282 describes an electromagnetic shield for use on a cell phone, microwave oven, computer, computer monitor, television set, radio, and a variety of other electronic devices. The shield comprises granular tourmaline distributed in a porous carier affixed to the device which produces negative ions (an opposing field) to combat electromagnetic radiation.

There remains a need to manage the exposure of electromagnetic radiation to the skin, especially the facial skin, without requiring clothing in close proximity with or against the skin. The present invention achieves these goals and meets the need for protection against electromagnetic radiation on facial skin in a manner that is consistent with the normal daily use of foundation or other similar skin care products.

### SUMMARY OF THE INVENTION

The present invention relates to a cosmetic or pharmaceutical composition for topical application to the skin which comprises a non-magnetic cationic component and a mineral enriched compound and a single domain magnetic component. The amounts of the mineral compound, the cationic component and the magnetic component are such that in combination, they shield the skin against an ambient electromagnetic field (EMF) by providing at least 80 percent dampening of the intensity of the emitted EMF. Therefore, the single domain particles of the magnetic component have E-M wave dampening properties when combined with the cationic component and the mineral compound, and the three components are present in the form of a cosmetic or pharmaceutical composition. By resonating with ambient E-M waves, these two components dampen variable E-M fields and provide protection against potential physiological nuisances on the skin surface.

The mineral enriched compound and the magnetic component containing single domain particles are both present in an E-M wave dampening effective amount. The cationic component comprises cations derived from alkali- or alkali earth metal ions, or zwitterions like amino-acids in the continuous phase of an oil in water emulsion. The cationic component in combination with the mineral enriched compound and the magnetic component provides a surprising effect in dampening or shielding EMF when topically applied to the skin. Accordingly, the present invention also includes methods related to the use of the present compositions, and particularly, the method of dampening the effect of ambient E-M fields on skin surface with a composition comprising 0.01 to 25.0 percent magnetite, 0.1 to 20.0 percent malachite and about 0.001 to 20.0 percent sodium chloride.

### DETAILED DESCRIPTION OF THE INVENTION

It has been surprisingly found that a combination of a mineral enriched magnetic component and a cationic component in a cosmetic or pharmaceutical composition provides effective shielding of the skin against ambient electromagnetic (E-M) waves and/or dampening of their intensity. Accordingly, the present invention is a cosmetic or pharmaceutical composition for protection against ambient E-M waves. The compositions comprise optimal amounts of ionic and magnetic components which can achieve a dampening in the intensity of the incoming emitted E-M field from the surrounding environment. The presence of the magnetic and ionic components is believed, while not wishing to be bound to any particular theory, to resonate with the emitted E-M waves in the surrounding environment, and thus, create a dampening effect of the oscillating E-M fields. Therefore, the compositions of the present invention provide protection for the skin against the physiological nuisances that may be associated with the emission of E-M waves from a variety of electronic devices.

The compositions contain optimal amounts of the mineral enriched magnetic component in combination with the cationic component to provide at least about 80 percent dampening of the intensity of the E-M field subjected to the skin. The cationic component is present in an amount of about 0.001 to about 20.0 percent. The combined ionic and magnetic components of the present invention can be used in any type of aqueous system or emulsion system such as, for example, oil in water emulsions, water in oil emulsions, triple emulsions, or other multi-phase emulsion systems. The cationic component can be any known type of cation. Examples of useful cations in the cationic component of the present invention include but are not limited to cations derived from sodium chloride and other salts, zwitterions such as amino acids, alkali-metal ions, alkali-earth ions. Preferably, the cation is present in the continous phase of an emulsion, and more preferably, the emulsion is an oil-in-water emulsion. More preferably, the ionic component is a cation derived from sodium chloride.

The magnetic component of the present invention comprises single domain magnetic particles in combination with a mineral type material. The single domain magnet particles are present in an amount of about 0.01 to about 25.0 percent. As used herein, a single domain magnet refers to a solid in which all the molecules are isotropically oriented. Generally, there exists within a given solid, a zone in which the molecules are isotropically oriented (i.e. in the same direction) and this can give origin to a magnetic field. Essentially, the creation of a single domain magnet/ferromagnet is not a property of an element, per se, but rather, is determined by the arrangement of electrons, allowing each element to behave like a little magnet. In some substances, individual magnetic moments behave cooperatively so that individual little magnets align themselves in the same direction. These substances strongly interact with a magnetic field because each domain rotates to align itself with the magnetic field and are named ferromagnetics after iron, the most magnetic material. Examples of other magnetic metals, include but are not limited to, cobalt, nickel, samarium, dysprosium, and gadolinium; and examples of magnetic metal alloys, include but are not limited to, non-ferro magnetic metal alloys containing copper, manganese, aluminum (e.g., Cu—65%, Mn—25%, and Al—10%;), and ferromagnetic materials which are known to one of ordinary skill in the art. The process for making a ferromagnet is known to one of ordinary skill in the art and can be made by magnetizing a ferromagnetic material.

Preferably, the mineral component as used in the present invention and herein is in combination with the ferrous based magnetic component of the present invention and includes, but is not limited to, SP ceramics, SPT ceramics, C ceramic, colloidal gold, fuchsite (a deep green variety of muscovite, “Brazilian Jade”) or granatite (malachite or staurolite), a mineral of a brown to black color occurring in prismatic crystals. The mineral component can also be ferrous-based and this term as used in the present specification means a mineral that may contain iron but not as a major component. Thus it is present in the mineral at comparable levels with other metals in the mineral or less than the amounts of at least one of the other metals. Preferably, the mineral component of the present invention is granatite, a silicate of aluminum, magnesium, and iron, as the sole single domain magnet. Typically, the particle size of the mineral component is less than 50 microns. For example, the fuchsite (potassium aluminum silicate) is less than 45 microns. The mineral component can also be a combination of different ceramics such as in a composite form. An example of a composite of different ceramics is in a material available under the word mark Zeropa (Zeropa S.R.L. Corp., Italy). More preferably, the mineral component comprises malachite. The mineral-enriched compound is present in the compositions of the present invention in an amount of about 0.001 to 20.0 percent.

Although the present invention can be in the form of an aqueous suspension or any type of emulsion, in a preferred embodiment the composition of the present invention is an oil-in-water emulsion. Surprisingly, it has been found that the oil-in-water emulsion has an enhanced effect on the protective properties of the compositions of the present invention. The oil-in-water emulsion contains optimal amounts of ionic and magnetic components for protection against environmental E-M waves which increases proportionally with an increase in the concentration of the ingredients. The compositions contain about 0.001 to 20.0 percent of the magnetic component, about 0.05 to 20.0 percent of the mineral-enriched compound and about 0.001 to 20.0 percent of the cationic component. This combination provides protection to the skin from alternating E-M fields when topically applied in a cosmetic composition. Further, the compositions can also contain sunscreens for protection outdoors against both E-M radiation and ultraviolet radiation.

Good protection, e.g., greater than 80 percent dampening of the intensity of the field of the incoming electromagetic radiation, in at least a 30 percent aqueous suspension is achieved with granatite, a magnetic component and sodium cations from a NaCl solution. Alternatively, a phosphate buffer solution can be used to provide cations. In an oil-in-water emulsion, specifically, the granatite is present in an amount of about 0.1 to about 5.0 percent, and the sodium chloride of the cationic component is present in an amount of about 0.01 to 2.0 percent.

The present invention also includes a method of shielding a skin surface against E-M waves in the surrounding environment. Other methods involving magnetic particles are known, such as for example, U.S. Pat. Nos. 6,033,655, 5,358,659, and 4,046,591. However, in each of these, the magnetic particles interact with a controlled and discretely directed magnetic field. It has not heretofore been known to incorporate single domain magnetic particles in combination with a non-magnetic ionic component randomly in a cosmetic or pharmaceutical composition. Further, the combination of these two components randomly in a composition has not been known to provide shielding protection against E-M waves when topically applied to the skin by reducing exposure of the E-M field on the skin. The reduction in exposure is between 50 to 90 percent.

The benefit of the combination of the cationic and magnetic components can be obtained in any type of topically applied composition such as especially, makeup composition, for example, foundations, eyeshadows, blushes, powders, lipsticks and lipglosses. In a preferred embodiment, the compositions of the present invention are used in a foundation. Preferably, the benefit of the present invention is obtained in a topically applied eye gel, pressed powder, cream or lotion composition, (e.g., moisturizing and other types of treatment products) wherein the product is applied to the skin for a therapeutic purpose, but also has the added advantage of protecting the skin from E-M waves in the surrounding environment. Finally, the topical product may be applied solely for the purpose of protecting the skin.

Thus, the compositions of the present invention also include a compatible carrier. By “compatible carrier” in the present specification and claims is meant any cosmetically acceptable carrier which is compatible with the ionic and magnetic components of the present invention. The carrier may contain one or more oil components. The oil component may be any pharmaceutically or cosmetically acceptable material which is substantially insoluble in water. These materials can be found for example in the CTFA International Dictionary of Cosmetic Ingredients as well as the U.S. Pharmacopoeia or other equivalent sources. Suitable oil components include, but are not limited to, natural oils, such as coconut oil; hydrocarbons, such as mineral oil and hydrogenated polyisobutene; fatty alcohols, such as octyldodecanol; esters, such as C12-15 alkyl benzoate; diesters, such as propylene glycol dipelargonate; triesters, such as glyceryl trioctanoate; sterol derivatives, such as lanolin; animal waxes, such as beeswax; plant waxes, such as carnauba; mineral waxes, such as ozokerite; petroleum waxes, such as paraffin wax; synthetic waxes, such as polyethylene; and mixtures thereof.

Suitable oil components may also be silicones. The silicone oil can be volatile or semi-volatile, or any combination thereof. Suitable volatile oils include cyclic and linear silicones, such as hexamethylcyclotrisiloxane, octamethylcyclotetrasiloxane, and decamethylcyclopentasiloxane or volatile linear dimethylpolysiloxanes; or mixtures thereof. Other volatile silicones include, but are not limited to, cyclomethicone; polymeric silicones such as dimethicone; alkylated derivatives of polymeric silicones, such as cetyl dimethicone and lauryl trimethicone; hydroxylated derivatives of polymeric silicones, such as dimethiconol; and mixtures thereof. The carrier comprises, in the composition as a whole, preferably silicone oil which is present in an amount of at least about 0.5 to about 60 percent by weight. Preferably, the compatible carrier is one that enhances the soft powdery feel of the composition. A particularly preferred carrier is a low volatile silicone oil.

The composition of the present invention, depending on the type of product it is used in, may include one or more film-forming agents. The use of a film-former can also improve the wear of the composition, and can confer transfer-resistance to the protective product. Examples of useful film-forming agents include natural waxes, polymers such as polyethylene polymers and copolymers of PVP, dimethicone gum, and resins, such as shellac, polyterpenes, and various silicone resins. A particularly preferred film-former is trimethylsiloxysilicate, used in an amount of from about 0.1 to about 20 percent by weight of the total composition.

In another embodiment of the present invention, the composition is a lipstick comprising the mineral, cationic and magnetic components in which case, it may also be desirable to incorporate one or more waxes in the composition. The term “wax” will be understood to encompass not only waxes in the traditional sense, i.e., those plant, animal or mineral waxes containing primarily esters of higher fatty acids and alcohols, free higher acids and alcohols, and saturated hydrocarbons, but also synthetic resinous products having a wax-like, i.e., hard, brittle, relatively non-greasy texture at room temperature, such as silicone waxes. Examples of suitable waxes include, but are not limited to, carnauba wax, candelilla wax, beeswax, microcrystalline wax, polyethylene, japan wax, synthetic wax, shellac wax, spermaceti, lanolin wax, ozokerite, bran wax, ceresin wax, bayberry wax, paraffin, rice wax, mink wax, montan wax, ouricoury wax, jojoba wax, and the like.

Additional preferred components of the cosmetic compositions of the invention include one or more pigments. Any cosmetically acceptable pigment, either organic, inorganic, or combinations thereof, can be used in the makeup compositions of the invention. The invention is further illustrated by the following non-limiting example.

### EXAMPLES

Anti-E-M field cream composition

| Material | Weight % |
|---|---|
| Distilled water | 60 |
| Polywax | 14.4 |
| Mineral oil | 5 |
| NaCl | 0.1 |
| Magnetite | 20 |
| Granatite and fuchsite | 0.5 |

Raw materials are suspended in water and layered between 2 glass coverslips. The coverslips are situated between a source of an E-M field and an electrometer. The reading of the electrometer when the glass coverslips contain a suspension or formula or when they are blanks permits the measurement of the dampening of the E-M field to be determined and the results are reported as percent protection.

Individuals are exposed for 15 minutes to a source of E-M field without cosmetic or with the formulas of the present invention topically applied to the facial skin in an amount of about 2 to 5 mg/cm2. After exposure, the electrical properties of the skin are measured using a gas discharge visualization camera which captures and analyzes photons generated from a corona discharge at the surface of the skin. In the absence of a topically applied cream, the exposure to E-M field introduces a reduction of more than 50% in the GDV measurement. Whereas, the presence of an anti-EMF cream limits to a few percent the change provoked by exposure to E-M field. Thus the E-M field exposure to the skin surface is shielded in the range of about 50 to 90 percent.

What we claim is:

1. An emulsion composition for electromagnetic field protection of the skin comprising an ambient electromagnetic wave dampening effective combination of a) at least one single domain magnetic component having a particle size of about 10 to 400 nm; b) a mineral-enriched compound; and c) a cation capable of forming an ionic diatomic molecule.

2. The composition of claim 1 in which said magnetic component is present in an amount of about 0.01 to 25.0 percent, said mineral-enriched compound is present in an amount of about 0.05 to 20.0 percent in combination with said cationic component which is present in an amount of about 0.001 to 20.0 percent all by weight of the composition.

3. The composition of claim 1 in which said mineral-enriched compound is selected from the group consisting of SP ceramics, SPT ceramics, C ceramic, colloidal gold, fuchsite and malachite.

4. The composition of claim 1 in which said mineral-enriched component comprises a malachite.

5. The composition of claim 1 in which said cationic component is selected from the group consisting of alkali-metal ions, alkali-earth ions and zwitterions.

6. The composition of claim 5 wherein said cationic component is in a continuous phase of a oil-in-water emulsion.

7. A method of dampening the effect of electromagnetic fields on skin cells comprising topically applying to the skin the composition of claim 1.

8. The method of claim 7 further comprising reducing exposure of the electromagnetic field on the skin by about 80 percent.

9. A method of dampening the effect of electromagnetic fields on skin cells comprising topically applying to the skin a composition comprising about 0.01 to 25.0 percent magnetite, 0.1 to 20.0 percent malachite and about 0.001 to 20.0 percent sodium chloride all by weight of the composition.

10. The method of claim 9 further comprising reducing exposure of the electromagnetic field on the skin by about 50 to 90 percent.
